(19) United States
(12) Patent Application Publication
(10) Pub. No.: US 2014/0120574 A1
(43) Pub. Date: May 1, 2014

(54) FLUORESCENT NITRIC OXIDE PROBES AND ASSOCIATED METHODS

(71) Applicant: Board of Regents, The University of Texas System, Austin, TX (US)

(72) Inventors: Eric V. Anslyn, Austin, TX (US); Youjun Yang, Shanghai (CN); Michelle A. Ivy, Austin, TX (US)

(21) Appl. No.: 13/909,345

(22) Filed: Jun. 4, 2013

Related U.S. Application Data
(60) Provisional application No. 61/656,416, filed on Jun. 6, 2012.

(51) Int. Cl. G01N 33/84; G01N 31/22

(52) U.S. Cl. CPC G01N 33/84; G01N 31/22
USPC 435/34; 546/172; 546/173; 436/116

(57) ABSTRACT

Nitric oxide probes comprising a compound represented by Formula I are provided. Methods of using this nitric oxide probes to detect nitric oxide are also provided.

### CROSS-REFERENCE TO RELATED APPLICATIONS

This application claims the benefit of U.S. Provisional Application No. 61/656,416, filed Jun. 6, 2012, which is incorporated by reference.

### BACKGROUND

The biological roles of nitric oxide (NO) have led chemists and molecular biologists to seek cellular imaging agents responsive to this species. The creation of such agents derives from the pivotal role of NO in vasodilation as an endothelial-derived relaxing factor (EDRF), and its function as a platelet aggregation inhibitor, neurotransmitter, antimicrobial agent, and due to its antitumor activity in cardiovascular, nervous, and immune systems. Although a variety of quantification techniques have been developed, fluorescence techniques are the most desirable because of their sensitivity, and high spatiotemporal resolution when combined with microscopy. Consequently, a number of fluorescent NO probes are available, but each is hampered by certain selectivity and/or synthetic limitations.

Currently, the most common approach for NO detection involves the use of ortho-diamino aromatics under aerobic conditions, which reacts with NO⁺ equivalent, presumably N₂O₃ to furnish fluorescent triazole derivatives. Turn-on fluorescence signals are achieved due to suspension of photoinduced electron transfer (PET). Examples using fluoresceins (such as DAF-2 DA), anthraquinones, rhodamines (such as DAR-4M AM), BODIPYs, and cyanines are documented. Such probes are among the current state of the art, yet severe limitations exist. First of all, in the presence of H₂O₂/peroxidase, OONO⁻, OH^{·}, NO₂^{·}, and CO₃^{·-}, the intrinsically electron rich diaminobenzene moiety is easily oxidized to an arylaminyl radical, which combines with NO and leads to triazoles. Second, dehydroascorbic acid (DHA) condenses with ortho-diamino aromatics and turns on the fluorescence of such probes. It was reported that 1 mM DHA yielded a fluorescence signal with the commercial NO probes DAF-2 DA, DAR-4M AM, comparable to 300 nM and 100 μM of NO respectively. Third, benzotriazoles are pH sensitive (pKₐ's≈6.69) near neutral pH. The pH sensitivity can be solved by methylation of one of the amines, however the reactivity of the probe toward DHA was undesirably enhanced.

The aforementioned limitations complicate NO detection using ortho-diamines. Hence, a series of metal ligand complexes for NO detection are also currently under development. For example, Cu^{II}(FL₅), displays a fluorescence enhancement upon exposure to NO and can be used as a cellular imaging agent. However, given a dissociation constant (K_{d}) of 1.5 μM and the presence other metal ions in physiological conditions, it is a concern that the complex will release cytotoxic Cu²⁺. Complexes with lower K_{d}'s were reported, though with decreased reactivity toward NO.

Most recently, single-walled carbon nanotubes (SWCN) wrapped with 3,4-diaminophenyl-functionalized dextrans were used for in vitro or in vivo studies. The NIR fluorescence of the SWCN is bleached by several reactive oxygen/nitrogen species, but at least NO does so more than others.

### SUMMARY

The present disclosure generally relates to nitric oxide probes. More particularly, the present disclosure relates to fluorescent nitric oxide probes and associated methods.

The present disclosure provides a nitric oxide probe that may be used to detect and/or image NO and associated methods. In one embodiment, a nitric oxide probe of the present disclosure comprises a compound that is represented by the following Formula I:

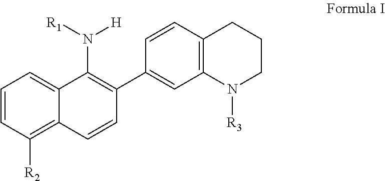

wherein R₁ is an alkyl group or H; R₂ is H, CN, SO₃⁻, sulfamoyl, alkyl substituted sulfamoyls, COO, carbamoyl, or alkyl substituted carbamoyls; and R₃ is a methyl or other alkyl.

The features and advantages of the present invention will be readily apparent to those skilled in the art upon a reading of the description of the embodiments that follows.

### DESCRIPTION

The present disclosure generally relates to nitric oxide probes. More particularly, the present disclosure relates to fluorescent nitric oxide probes and associated methods.

The present disclosure provides a nitric oxide probe that may be used to detect and/or image NO. In one embodiment, a nitric oxide probe of the present disclosure comprises a compound that is represented by the following Formula I:

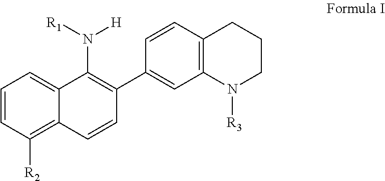

wherein R₁ is an alkyl group or H, R₂ is H, CN, SO₃⁻, sulfamoyl, alkyl substituted sulfamoyls, COO⁻, carbamoyl, or alkyl substituted carbamoyls, R₃ an alkyl group or methyl.

While not wishing to be bound to any particular theory, it is believed that upon introduction of nitric oxide under aerobic conditions to a nitric oxide probe of the present disclosure, a nitrosation reaction occurs to yield a nitrosamine, which is subsequently scavenged via an electronic aromatic substitution reaction. The product of this reaction has an extended conjugation system in comparison to the initially synthesized compound and displays red shifted spectral properties, which are easily detected through fluorescence.

One of the many advantages of the present disclosure, many of which are not discussed herein, is that the nitric oxide probes of the present disclosure are highly selective and have not been found to interfere with reactive oxygenated species, reactive nitrogen species, ascorbic acid (AA), and dehydroascorbic acid (DHA). The probes of the present disclosure have also been shown to successfully respond to nitric oxide within cellular media. Due to the various roles of nitric oxide in the body, a sensing method utilizing a nitric oxide probe of the present disclosure can be applied to study any of the biological pathways where nitric oxide may be involved. In addition to the advantage of high specificity, a nitric oxide probe of the present disclosure is also advantageous due to its facile synthesis, low pH dependence, and fast reaction kinetics.

In one embodiment, a method of the present disclose comprises contacting a sample with a nitric oxide probe comprising a compound represented by Formula I, and detecting emitted fluorescence from the nitric oxide probe. In some embodiments, the detection of emitted fluorescence involves the detection of a turn on fluorescence signal from a dark background at the longer wavelength upon nitric oxide addition, rather than a fluorescent signal fluctuation from a non-zero background seen by most nitric oxide detecting systems. Highly electron rich ortho-diamino aromatics were avoided so as to impede general oxidation by other reactive oxygen/nitrogen species and condensation with ascorbic acid (AA) analogs. In some embodiments, a spectrofluorometer may be used to detect emitted fluorescence from a nitric oxide probe.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

What is claimed is:

1. A nitric oxide probe comprising a compound represented by the following Formula I: 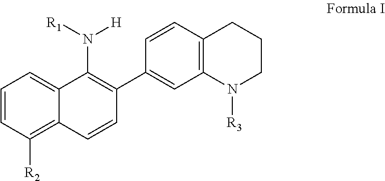
wherein R₁ is an alkyl group or H; R₂ is H, CN, SO₃⁻, sulfamoyl, alkyl substituted sulfamoyls, COO, carbamoyl, or alkyl substituted carbamoyls; and R₃ is a methyl or other alkyl.

2. A method of detecting the presence of nitric oxide in a sample comprising:
contacting a sample with the nitric oxide probes of claim 1; and
detecting emitted fluorescence from the nitric oxide probe, wherein the emitted fluorescence indicates the presence of nitric oxide in the sample.
